(19) United States
(12) Patent Application Publication
(10) Pub. No.: US 2006/0212108 A1
(43) Pub. Date: Sep. 21, 2006

(54) System for treatment of extensive obliterative vascular diseases

(75) Inventor: Michael Tittelbach, Nurnberg (DE)

Correspondence Address: HAHN LOESER & PARKS, LLP, One GOJO Plaza, Suite 300, AKRON, OH, 44311-1076, US

(73) Assignee: BIOTRONIK VI Patent AG, Baar (CH)

(21) Appl. No.: 11/376,925

(22) Filed: Mar. 16, 2006

(30) Foreign Application Priority Data
Mar. 17, 2005 (DE) 10 2005 013 221.9

(51) Int. Cl. A61F 2/82

(52) U.S. Cl. 623/1.15; 623/1.42

(57) ABSTRACT

A system for treating extensive obliterative vascular diseases, in which the severity of the disease is different in different vascular sections, comprises: one or more biocorrodible stents arranged with a distance between them in sections of the diseased vessel that have been dilated by transluminal angioplasty, and a device adapted for release of an active ingredient in the lumen or in the vascular tissue, designed (i) to release the active ingredient in the lumen of the diseased vessel, whereby the active ingredient is released in a vascular section at the greatest distance proximally in the direction of blood flow in the vessel; or (ii) to release the active ingredient in the vascular tissue whereby the active ingredient is released in one or more vascular sections of the vessel to be treated so that the active ingredient can spread throughout the area of the diseased vessel through diffusion.

### TECHNICAL FIELD

The invention relates to a system for treatment of extensive obliterative vascular diseases.

### BACKGROUND OF THE INVENTION

With many obliterative vascular diseases, purely systemic therapy is impossible or yields only limited success. For example, in acute angina pectoris or when arteries are completely occluded, it is usually necessary to dilate the vessel in the affected area. This may be accomplished in particular by percutaneous transluminal angioplasty (PTA, often referred to as balloon angioplasty) in which the vessel is dilated in the diseased vascular section.

In most cases, the dilated vascular section must be stabilized by implanting a stent to prevent renewed re-occlusion due to vascular stenosis. Stents are usually designed as permanent implants, i.e., remaining permanently in the patient's body, frequently without being surgically removable after fusion with the vascular wall. It has now been found that the permanent presence of such an implant may itself be the starting point for microbiological processes leading to re-occlusion of the vessel (restenosis). One proposal for remedying this situation has been to coat stents with medication to counteract or prevent the underlying microbiological processes. Production of medication-coated implants requires the development and implementation of specific procedures for production, validation, quality assurance and approval. In other words, production of such medication-coated systems is time-consuming and especially expensive in terms of development and production.

U.S. Pat. No. 6,547,803 B2 discloses a modified balloon catheter with which an active ingredient can be injected into vascular tissue, in particular into the adventitia of the vascular wall. To this end, the catheter has a microneedle in an expandable area which moves outward approximately perpendicularly and, in doing so, penetrates through the vascular wall when the balloon is inflated. The needle is connected to an active ingredient depot, so that the active ingredient can be introduced into deeper layers of tissue of the vascular wall. It has been found that the active ingredient is distributed within the vascular wall by diffusion. It is thus possible to introduce a medication treatment into the vascular tissue over an area extending beyond the injection site.

U.S. Patent Application No. 2004/0010309 A1 describes a method and a system for supplying a liquid substance to the surrounding tissue around a blood vessel, especially a coronary artery. This system comprises a catheter of the type described in U.S. Pat. No. 6,547,803 B2, with an injection needle by which the liquid substance is introduced into the tissue. In addition, the system comprises a stent having a structure for absorbing the liquid substance injected. According to this method, the substance is injected into the vascular tissue near the supporting structure, absorbed by the absorptive structure of the supporting structure and released again after absorption. The supporting structure is implanted before, during or after injection of the substance.

Stents may also be made of a biocorrodible material. This approach is based on the finding that the supporting function of the implant need usually be maintained for only a few weeks to a few months during which the treated vascular section has usually largely regenerated. It would be conceivable to coat biocorrodible implants with medications which counteract any microbiological processes induced by the surgical procedure which could lead to restenosis. However, findings about coating permanent implants with medication cannot be transferred easily to biocorrodible implants because special problems occur in this regard. For example, a medication coating, whether as a pure active ingredient or embedded in a suitable matrix, has a considerable influence on the corrosive processes leading to degradation of the implant. Conversely, the corrosive processes and the degradation products thus formed influence the elution and stability of the medication. These interactions are complex and can be predicted only in the general trends. Procedures for production, validation, quality assurance and approval of a biocorrodible stent are complex accordingly and are associated with a considerable cost.

### SUMMARY OF THE INVENTION

An aspect of the invention is to provide a system for treatment of extensive obliterative vascular diseases which relies on stents made of biocorrodible materials while also permitting treatment of the diseased vessel with medication. Both aspects of treatment should be taken into account to such an extent that interfering interactions are prevented or at least largely suppressed.

This aspect is achieved by the inventive system for treatment of extensive obliterative vascular diseases in which the severity of the disease differs in different vascular sections. The system comprises:
one or more biocorrodible stents arranged with a distance between them in vascular sections of the diseased vessel that have been dilated by transluminal angioplasty, and
   a device adapted for releasing an active ingredient in the lumen or in the vascular tissue, designed
(i) to release the active ingredient in the lumen of the diseased vessel, whereby the active ingredient is released in a vascular section at the greatest distance proximally in the direction of blood flow in the vessel; or
(ii) to release the active ingredient in the vascular tissue, whereby the active ingredient is released in one or more vascular sections to be treated so that the active ingredient can spread throughout the area of the diseased vessel through diffusion.

The invention is based on the finding that improved treatment and thus improved treatment results can be obtained by separating the requirements of distribution of the active ingredient in the lumen or tissue of the diseased vessel, in particular also deep into the vascular tissue, and accurate dosing, possibly adapted to the individual patient's needs, from the requirements regarding the mechanical properties of the biocorrosive stent, (e.g., degradation behavior, accessibility from the side, geometric optimization for maximum supporting effect with minimum coverage of the vascular wall through the use of alloplastic material). Consistent separation between the corroding implant and the device for active ingredients also permits a definite simplification of the procedures for production, validation, quality assurance and approval of the biocorrosive stents because it is not necessary to coat the stents with medication. Adverse interactions between the active ingredient and/or a matrix holding the active ingredient and the corrosive processes in degradation of the stent and/or the degradation products of the biodegradation can be prevented or at least definitely reduced.

According to a preferred embodiment of the invention, the stent is made of a biocorrosive metal alloy, in particular a biocorrosive magnesium alloy. The biocorrosive magnesium alloy may also preferably be an alloy having the composition:
yttrium: 3.7-5.5 wt %
rare earths: 1.5-4.4 wt % and
remainder: <1 wt %,
   whereby magnesium makes up the remainder of the alloy to a total of 100 wt %. Stents made of the aforementioned materials are characterized by their advantageous mechanical properties in comparison with synthetic biocorrodible polymers or those produced from synthetic or natural sources. In addition, biocorrosive magnesium alloys in particular can also be processed easily and the degradation products of the alloys are tolerated very well in the body.

According to another preferred embodiment of the invention, the device is a balloon catheter designed to release an active ingredient or an implant designed to release an active ingredient.

Furthermore, it is preferable for the device to be arranged in such a way that the release of the active ingredient into the vascular lumen occurs at least approximately 1 cm, in particular at least approximately 5 cm away from the nearest stent. In addition, it is preferable for the device to be arranged in such a way that the active ingredient is released in the vascular tissue at least approximately 0.2 cm, especially at least approximately 0.5 cm away from the nearest stent. This takes into account the fact that the active ingredient should be released in relative proximity to the diseased vascular areas but not directly at the stent in order to avoid or at least minimize interactions between the active ingredient thereby released and the products of the corrosive process in the degradation of the stent.

A respective inventive method for treating extensive obliterative vascular diseases in which the severity of the disease differs in various vascular sections includes the following steps:
(a) providing a device adapted to release of an active ingredient in the vascular lumen or vascular tissue,
(b) dilation of individual vascular sections by transluminal angioplasty,
(c) insertion of one or more biocorrodible stents into the vascular sections dilated by transluminal angioplasty, and
(d) releasing the active ingredient by means of the device before, during or after step (c), whereby
   (I) the active ingredient is released into the vascular lumen in a vascular section situated most proximally in the direction of blood flow of the vessel;
   or whereby
   (II) the active ingredient is released in the vascular tissue in one or more vascular sections of the vessel to be treated so that the active ingredient can spread through diffusion in the area of the diseased vessel.

According to this method, extensive obliterative vascular diseases can be treated in a differentiated manner according to the severity of the disease in individual vascular sections. Only the areas dilated by transluminal angioplasty are supported by a stent to reduce restenosis through obstruction of the vessel. In the other vascular sections, treatment occurs only through the release of active ingredient. This method is especially suitable for treating heavily calcified vascular sections.

This method may preferably be performed with the release of the active ingredient into the tissue, especially into the vascular adventitial tissue.

This method and this system are suitable in particular for the release of active ingredients in conjunction with biocorrosive stents made of a magnesium alloy, especially having the preferred composition given above. Active ingredients that are not stable in an alkaline medium or are deactivated by complexing with magnesium ions are especially preferred. In this connection, the active ingredients paclitaxel, sirolimus and pimecrolimus are preferred.

It is also preferred if the device is removed after the active ingredients are released.

### BRIEF DESCRIPTION OF THE DRAWINGS

FIG. 1 shows a detail of a blood vessel containing the inventive system in a first variant; and

FIG. 2 shows a detail of a blood vessel containing the inventive system in a second variant.

### DETAILED DESCRIPTION OF THE INVENTION

The invention relates to a system and a method for treatment of extensive obliterative vascular diseases in which the severity of the disease in various vascular sections differs. The inventive system has already been outlined briefly and includes a certain arrangement of one or more biocorrodible stents in the vessel and a device adapted in a certain manner for the release of an active ingredient in the vascular lumen or tissue.

The term “obliteration” is understood to refer mainly to obstruction of the inside diameter of a vessel, in particular due to inflammatory processes (pleuritis, pericarditis, endangiitis), a neoplasm or thrombus.

“Obliterative vascular diseases” (also known as occlusive diseases) in the sense of the invention include:
(i) arterial: arteriosclerosis, diabetic angiopathy, inflammatory vascular diseases (arteritis, endangiitis), thromboses, emboli
   a) in the area of the extremities: peripheral arterial occlusive disease
   b) viscerally (e.g., the carotid artery, coronary heart disease, renal artery stenosis, occlusion of larger aortic branches); and
(ii) venous: deep vein thrombosis, thrombophlebitis, phlebitis.

The term “vessel” in the sense of the present invention is understood to refer to the totality of the arterial and venous blood vessels, including the vessels of the terminal vascular bed which, together with the heart, form a functional unit. Such a vessel has an inner lumen through which blood flows. The vessel is defined geometrically by its vascular, wall which extends from the inside, starting from the intima, to the adventitia.

The term “extensive” in the present case is understood to refer to a longer section of the vessel which is divided into at least two vascular sections with different severities of the obliterating disease. At least one vascular section is so severely damaged that transluminal angioplasty for dilation is indicated and there is at least one vascular section in which this is not necessary. The “severity of the disease” in the inventive sense is thus based on whether or not the diseased vascular section must be dilated by transluminal angioplasty to restore its functionality. To adjust the dosage of the active ingredient to be released, the severity of the disease may also have a more differentiated influence, i.e., the extent of the pathological changes in the vessel occurring due to the disease can be taken into account in adjusting the dosage of the active ingredient. An extensive obliterative vascular disease in the sense of the invention preferably extends over a length of 5 to 70 cm, especially in the range of 10 to 50 cm, of the vessel.

“Transluminal angioplasty” refers mainly to a closed percutaneous (=percutaneous transluminal) dilation of vessels with the help of coaxial catheters or—as is usually the custom today—by means of a balloon catheter, possibly as an additional measure in vascular surgery. This also includes percutaneous transluminal coronary angioplasty (PTCA) for dilation of stenoses of the coronary vessels (=coronary angioplasty). Angioplasty has recently also been possible by laser techniques and various other techniques.

The phrase “device adapted for release of an active ingredient in the lumen or vascular tissue” in the sense of the present invention is understood to refer to a device with which one or more active ingredients can be released actively or passively into the lumen or vascular tissue. Active devices include actuators whose operation conveys the active ingredient to the site of release. Passive devices include structural elements which permit the release of active ingredients through diffusion or as a result of corrosive processes.

An active device may preferably be a balloon catheter designed to release active ingredients. Such a balloon catheter may be coated with the active ingredient, for example, or designed so that it conveys the active ingredient out of a depot to the desired site of release on inflation of the balloon. A balloon having a microneedle as described in U.S. Pat. No. 6,547,803 B2 and U.S. Patent Application No. 2004/0010309 A1 is especially preferred. The contents of these two documents are incorporated herein by reference.

A passive device may preferably be designed as an implant which is anchored in the vascular wall. The implant contains structural elements which permit the active ingredient to be released by diffusion or as a result of corrosive processes. For example, these structural elements include flat sections with indentations, pockets or the like in which the active ingredient is embedded in a form of administration suitable for this application. If the flat sections are arranged in the vascular lumen, the active ingredient is dissolved out, i.e., entrained by the blood flowing past it. If the flat sections are on the vascular wall, then diffusion processes tend to be dominant and the active ingredient can penetrate into the vascular wall tissue. The passive devices may also be designed to introduce the active ingredient into deeper sections of the vascular wall—for example, by an active ingredient depot that is connected to a microcannula which penetrates through the vascular wall when the device is placed in the vessel. Then the active ingredient can be released by diffusion. The passive devices are preferably made of a biocorrosive material.

An “active ingredient” in the sense of the invention may be a substance of animal, vegetable or synthetic origin, which, when administered in a suitable dosage as a therapeutic agent, serves to influence states or functions of the body, as a substitute for active ingredients or bodily fluids naturally produced by the human or animal body and to eliminate or render harmless disease pathogens, parasites or exogenous substances. The active ingredients that are used are thus pharmaceutical drugs and serve in particular to prevent and treat obliterative vascular diseases. The active ingredients used for the purposes of release in the vascular tissue are preferably lipophilic because lipophilic active ingredients are distributed especially uniformly through diffusion in the tissue. Several active agents may also be administered simultaneously or with a time lag with this system and the respective method.

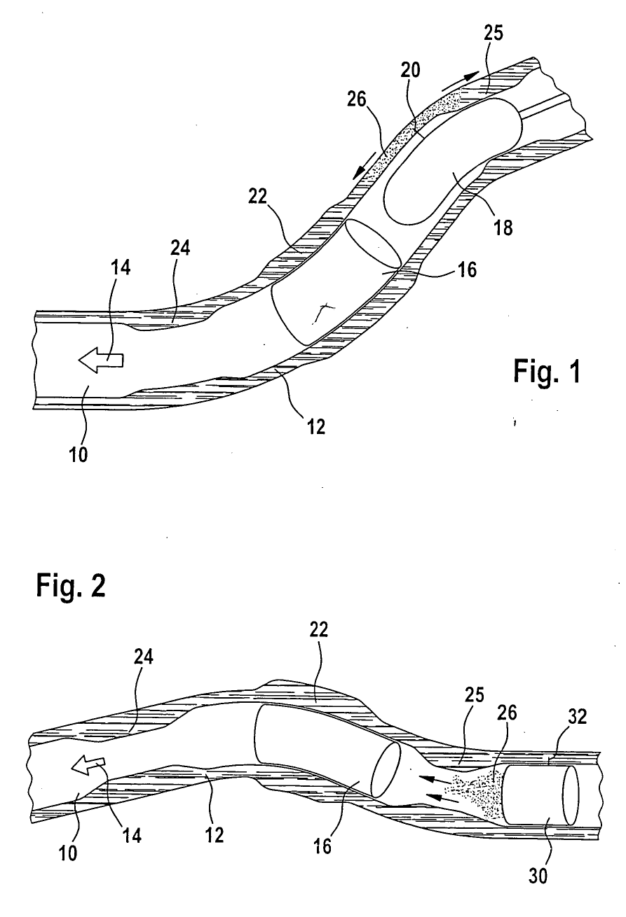

FIG. 1 shows in highly schematized form a detail of a blood vessel 10 in a half section through its vascular wall 12. The direction of flow of the blood in the vessel 10 is indicated by the arrow 14.

The system for treatment of the vessel 10 comprises a biocorrodible stent 16 and a device for release of the active ingredient in the form of a balloon catheter 18 with a microneedle 20.

The biocorrodible stent of made of a magnesium alloy with the composition 3.7-5.5 wt % yttrium, 1.5-4.4 wt % rare earths and remainder <1 wt % whereby magnesium makes up for the remainder of the alloy to a total of 100 wt %. The stent 16 is arranged in a vascular section 22 of the vessel 10 which was initially occluded as the result of a stenosis and was then dilated by transluminal angioplasty. The stent 16 can be brought to the implantation site using an application system, e.g., a balloon catheter and be readjusted there by expansion of its function of supporting the vascular wall 12 in the area of the vascular section 22.

Two other sections 24, 25 of the vessel 10 are already obstructed by calcification. However, the previous extent of the deposits has not previously justified transluminal angioplasty and subsequent implantation of another stent in the sections 24, 25. However, other stents may be arranged in the vessel to be treated.

The balloon catheter 18 injects an active ingredient 26 into the vascular wall 12 through its microneedle 20 when the balloon is inflated. The active ingredient is released approximately over a region of 3 to 5 cm in front of the stent 16. The active ingredient 26 released in the vascular wall 12, in particular in the adventitia is distributed by diffusion in the vascular wall 12 in the longitudinal direction (indicated by the two small arrows) as well as in the circumferential direction of the vessel (not shown here). Accordingly, the active ingredient 26 first reaches the vascular section 22 in the area of the stent 16 in the distal direction of blood flow and then in the proximal direction of blood flow it reaches section 25 and can manifest its pharmacological action there.

The quantity of injected active ingredient of course depends on the properties of the active ingredient itself (e.g., solubility, rate of degradation in the body, diffusion rate) and its mechanism of action in the body. However, the quantity of active ingredient is specified so that there will still be a sufficient concentration of the active ingredient even in vascular section 24.

After injection of the active ingredient 26, the balloon catheter 18 is deflated and removed from the blood vessel 10 again. The stent 16 remains in the body, but is gradually degraded by corrosive processes.

FIG. 2 shows a vessel 10 damaged by an extensive obliterative disease which is essentially similar to that illustrated in FIG. 1. To this extent, reference is made to the preceding discussion and the same reference numerals are used for the same or very similar features.

The system depicted in FIG. 2 differs from that in FIG. 1 in that the device for release of the active ingredient 26 is an implant 30. The implant 30 has a tubular contour through which blood flows. In the interior, the implant 30 has components 30 to which the active ingredient 26 is bound in pure form or embedded in a suitable matrix, these components being designed so that the blood can dissolve or entrain the active ingredient 26 as it flows past. The implant is arranged in a vascular section 32 that is the most proximal from the diseased part of the vessel 10 in the direction of blood flow. The implant 30 is preferably made of a biodegradable material, thereby eliminating the need for any subsequent surgical removal.

1. A system for treating extensive obliterative vascular diseases in which the severity of the disease in different vascular sections differs, the system comprising:
one or more biocorrodible stents arranged with a distance between them in sections of the diseased vessel that have been dilated by transluminal angioplasty, and
a device adapted for release of an active ingredient in the lumen or in the vascular tissue, designed
(i) to release the active ingredient in the lumen of the diseased vessel, whereby the active ingredient is released in a vascular section at the greatest distance proximally in the direction of blood flow in the vessel;
or
(ii) to release the active ingredient in the vascular tissue whereby the active ingredient is released in one or more vascular sections of the vessel to be treated so that the active ingredient can spread throughout the area of the diseased vessel through diffusion.

2. The system according to claim 1, wherein the one or more biocorrodible stents are made of a biocorrosive metal alloy.

3. The system according to claim 2, wherein the one or more stents are made of a biocorrosive magnesium alloy.

4. The system according to claim 3, wherein the biocorrosive magnesium alloy is an alloy of the composition
yttrium: 3.7-5.5 wt %
rare earths: 1.5-4.4 wt % and
remainder: <1 wt %,
where magnesium accounts for the remaining amount of the alloy to a total of 100 wt %.

5. The system according to claim 1, wherein the device is a balloon catheter designed for releasing an active ingredient.

6. The system according to claim 1, wherein the device is an implant designed for releasing an active ingredient.

7. The system according to claim 1, wherein the device is arranged so that the active ingredient is released into the vascular lumen at least approximately b 1 cm away from the nearest stent.

8. The system according to claim 1, wherein the device is arranged so that the active ingredient is released into the vascular lumen at least approximately 5 cm away from the nearest stent.

9. The system according to claim 1, wherein the device is arranged in such a way that the active ingredient is released in the vascular tissue at least approximately 0.2 cm away from the nearest stent.

10. The system according to claim 1, wherein the device is arranged in such a way that the active ingredient is released in the vascular tissue at least approximately 0.5 cm away from the nearest stent.

11. A method for treating extensive obliterative vascular diseases in which the severity of the disease in different vascular sections is different in different vascular sections, comprising the steps:
(a) providing a device adapted to release of an active ingredient in the vascular lumen or vascular tissue,
(b) dilating individual vascular sections by transluminal angioplasty,
(c) inserting one or more biocorrodible stents into the vascular sections dilated by transluminal angioplasty, and
(d) releasing the active ingredient by means of the device before, during or after step (c), whereby
(I) the active ingredient is released into the vascular lumen in a vascular section situated most proximally in the direction of blood flow of the vessel;
or whereby
(II) the active ingredient is released in the vascular tissue in one or more vascular sections of the vessel to be treated so that the active ingredient can spread through diffusion in the area of the diseased vessel.

12. The method according to claim 11, wherein the active ingredients released are paclitaxel, sirolimus and pimecrolimus.
